(19) United States
(12) Patent Application Publication
(10) Pub. No.: US 2019/0110776 A1
(43) Pub. Date: Apr. 18, 2019

(54) Methods for Computing Coronary Physiology Indexes Using a High Precision Registration Model

(71) Applicants: Bo Yu, Harbin (CN); Haibo Jia, Harbin (CN); Sining Hu, Harbin (CN); Jiannan Dai, Harbin (CN); Lei Xing, Harbin (CN); Chenyang Xu, Beijing (CN); Zhao Wang, Beijing (CN); Shuai Zhang, Beijing (CN)

(72) Inventors: Bo Yu, Harbin (CN); Haibo Jia, Harbin (CN); Sining Hu, Harbin (CN); Jiannan Dai, Harbin (CN); Lei Xing, Harbin (CN); Chenyang Xu, Beijing (CN); Zhao Wang, Beijing (CN); Shuai Zhang, Beijing (CN)

(73) Assignee: Panorama Scientific Co. Ltd, Beijing (CN)

(21) Appl. No.: 16/153,825

(22) Filed: Oct. 8, 2018

(30) Foreign Application Priority Data
Oct. 9, 2017 (CN) 201710927703.2

(51) Int. Cl. A61B 8/08; A61B 5/00; A61B 8/06; A61B 5/027; A61B 8/12; A61B 6/12; G06T 7/33; G06T 7/70; G06T 7/20; G06T 7/00; G16H 30/20

(52) U.S. Cl. CPC A61B 8/5223; A61M 25/0108; A61B 5/0035; A61B 5/0084; A61B 8/06; A61B 5/027; A61B 8/12; A61B 6/12; A61B 8/5261; G06T 7/344; G06T 7/70; G06T 7/20; G06T 7/0012; G16H 30/20; A61B 5/0066

(57) ABSTRACT

This invention describes methods to compute coronary physiology indexes using a high precision registration model, which consists of acquiring coronary angiography images of coronary vessels, performing intravascular imaging, and registering the coronary angiography images with intravascular images to create a high precision registration model, based upon which the coronary flow, fractional flow reserve (FFR) and index of microcirculation resistance (IMR) can be computed. The methods described in this invention to compute coronary flow, FFR, IMR are based on both coronary angiography and intravascular images, and the accuracy is better than those derived from coronary angiography alone or intravascular imaging alone, and have high practical values.

### FIELD OF THE INVENTION

This invention relates to the field of medicine, and in particular relates to methods for computing coronary physiology indexes using a high precision registration model.

### BACKGROUND OF THE INVENTION

Coronary artery disease due to coronary artery stenosis is a severe disease affecting people's health, and accurate diagnosis and treatment of coronary artery disease is of paramount importance. In general, moderate or severe coronary artery disease requires coronary angiography (CAG) for making a diagnosis. Although almost all coronary interventions require coronary angiography, coronary angiography is a projection based imaging technique with relatively low resolution, and is based on limited angular projections, therefore the reconstruction of three dimensional vasculature from coronary angiography has limited accuracy. Intravascular imaging methods such as intravascular ultrasound (IVUS) and intravascular optical coherence tomography (OCT) have better precision and accuracy compared with CAG. They can provide rich morphological information of vascular lumen and walls, and help physicians make treatment decisions. For instance, IVUS can detect calcified plaques within the vessel wall, which should be treated using rotablators or cutting balloons if severe enough before percutaneous coronary intervention. IVUS and OCT are typically realized using interventional imaging catheters.

The clinical representation of coronary artery stenosis is myocardial ischemia. However, pure morphological assessment based on vascular imaging cannot provide direct diagnostic evidence of clinical physiology, and the relationship between morphology and physiology is not necessarily straightforward and clear. Early evidence showed that the maximum stenosis percentage of a vessel measured by CAG or IVUS is unable to accurately predict the downstream myocardial ischemia, although there exists some correlations. This indicates that the vascular function may not be determined by a single morphological parameter, but by multiple parameters in a complex manner.

Fractional flow reserve (FFR) is a method to directly measure the vascular function, and is defined as the ratio between the maximum blood flow in a diseased coronary artery and the maximum blood flow in a normal coronary artery. FFR can provide direct valuable diagnostic information, and generally revascularization is indicated if FFR≤0.8, or not recommended if FFR>0.8. A series of clinical studies have proven that the diagnosis and treatment based on FFR can improve patients' post-procedure outcome and reduce medical expenses. The measurement of FFR typically requires an invasive pressure wire.

Functional assessment by FFR and morphological measurements by intravascular imaging can provide complementary information about the vascular pathology, and ideally should be both provided to physicians. However, the pressure wire used by FFR and the imaging catheter used by IVUS or OCT are not the same instrument, and therefore simultaneous use of both technologies would result in an increase of cost and operational complexity.

Generally speaking, the morphological structure of human organs and tissues determines the function, and the function reflects the structure. Therefore, it is meaningful to derive functional parameters from structural imaging, or infer morphological structures from functional measurements. This area has attracted considerable research interest from early days. Patent CN105326486A “Method and system for calculating blood vessel pressure difference and fractional flow reserve” disclosed a computer model to compute FFR based on coronary angiography images. Patent CN103932694A “Method and device for accurately diagnosing FFR” disclosed a computer model to calculate FFR based on Computed Tomography (CT) and ultrasonocardiogram using computational fluid dynamics (CFD) theories. The two inventions listed above both derived FFR from structural imaging, typically realized by calculating the pressure difference between the distal and proximal end of the vessel. Generally speaking, the pressure difference is determined by two factors, the coronary blood flow and the vessel area function. However, the resolution of both CAG and CT are relatively low, about 0.5 mm, which is inadequate to provide accurate measurement of coronary arteries with a diameter approximately between 2-4 mm. Therefore, the resulting blood flow and vessel area distribution cannot be measured accurately, and that the accuracy of FFR calculation cannot be assured.

Patent US20130072805A1 disclosed an apparatus and method to acquire and measure lumen morphology and vascular resistance, in particular related to a method to compute FFR indirectly using OCT images. The resolution of OCT imaging is high, about 0.02 mm, therefore it is able to measure the vessel area accurately. However, OCT imaging alone is unable to measure blood flow effectively, therefore this method adopted an average flow parameter from normal population, and the accuracy of the computed result can be low.

Myocardium ischemia can be either caused by coronary artery stenosis, or by high microcirculation resistance. FFR can only reflect coronary artery stenosis, but is unable to measure microcirculation resistance. Although coronary flow reserve (CFR) can measure the total resistance from both coronary artery and microcirculation during maximum achievable blood flow, it is unable to tell whether the ischemia is from the stenosis of epicardial vessels or from diseased microcirculation. Index of microcirculation resistance (IMR) is a new index proposed by Fearon et al (Fearon W F, Balsam L B, Farouque H M O, et al. Novel index for invasively assessing the coronary microcirculation. Circulation, 107(25), 2003), and is defined as the distal coronary pressure divided by coronary flow, and is able to accurately assess the microcirculation resistance by excluding the effect from the proximal coronary artery stenosis. Therefore in the clinical settings, the ideal scenario is to obtain FFR and IMR simultaneously, and comprehensively assess the resistance from both the coronary arteries and microcirculation and adopt proper treatment measures. Currently, the typical method to assess IMR is to measure the distal coronary pressure via a pressure wire during maximal hyperemia, and measure the approximate coronary flow using thermodilution by calculating the mean transit time of a bonus of saline through the coronary artery at room temperate, and the ratio between the pressure and the flow is IMR.

The functional feedback provided by FFR/IMR and the morphological information offered by imaging complement each other. Ideally, it is desirable to obtain both information. However, currently there is no such technology or apparatus that can measure both function and morphology. Conventional methods require multiple instruments, which could prolong the coronary catheterization time, increase the patient expenses, and accumulate the risks from multiple invasive measurements.

### BRIEF SUMMARY OF THE INVENTION

The purpose of the current invention is to address the problems of high complexity, high cost and low precision associated with the current methods for measuring coronary artery parameters, and propose a more precise method to computationally calculate coronary blood flow, fractional flow reserve, and index of microcirculation resistance.

For the above purpose, the technical approach of the current invention comprising: A method to compute coronary physiology indexes based on a high precision registration model. In one embodiment, the method acquires coronary angiography and intravascular images of coronary arteries, and register the coronary angiography with intravascular images into a high precision registration model, and compute coronary blood flow, FFR and IMR from the high precision registration model.

In one embodiment, the method of registering the coronary angiography with intravascular images into a high precision registration model is realized by placing a radio opaque marker that can be moved together with the probe inside the intravascular imaging catheter, tracking the marker's position and pullback trajectory, locating the positions of the intravascular images in the corresponding coronary angiography, and finally matching the intravascular imaging with coronary angiography by signal synchronization and processing.

One embodiment of the method to compute coronary blood flow based on the high precision registration model is to select a segment of vessel from the high precision registration model, and measure the transit time of the contrast traveling through the vessel segment, and compute the lumen volume of the vessel segment in the high precision registration model, and calculate blood flow based on equation (1):

$$Q = \frac{V}{Δ T} \qquad ( 1 )$$

Where Q is the coronary blood flow, ΔT is the contrast transit time, V is the lumen volume.

The said lumen volume V is computed based on the morphological parameters measured from intravascular imaging using the high precision registration model.

The method to measure the contrast transit time ΔT inside the vessel segment is to inject contrast at the proximal end of the vessel, and record the time of the first frame of the coronary angiography T₁, the time of the second frame T₂, and so forth. Then the contrast transit time is obtained as ΔT=T_{d}−Tₚ, where Tₚ is the contrast arriving time at the proximal end of the vessel segment, and T_{d} is the contrast arriving time at the distal end of the vessel segment.

The said fractional flow reserve is obtained from coronary blood flow, together with the vascular morphological parameters measured using the high precision registration model.

The said index of microcirculation resistance is calculated from fractional flow reserve.

The said index of microcirculation resistance is calculated using equation (2):

$$IMR = FFR \times \frac{{P}_{a}}{Q} \qquad ( 2 )$$

Where FFR is fractional flow reserve, Pₐ is mean arterial pressure, Q is blood flow.

The said intravascular imaging consists of intravascular ultrasound, intravascular optical coherence tomography, and combined use of intravascular ultrasound and intravascular optical coherence tomography.

The benefits of this invention are:

The methods to compute coronary blood flow, fractional flow reserve, and index of microcirculation resistance described in this invention are based on a high precision registration model with joint use of coronary angiography and intravascular imaging, and have higher accuracy than those derived from coronary angiography alone or intravascular imaging alone, and have high practical values.

### BRIEF DESCRIPTION OF DRAWINGS

FIG. 1 is an illustration of the vessel model obtained from the high precision registration of coronary angiography and intravascular imaging.

FIG. 2 is an illustration of the transit time ΔT of the contrast traveling through the vessel segment.

FIG. 3 is a schematic of the relationship between the errors of pressure difference calculation and the errors from diameter measurements.

### DETAILED DESCRIPTION OF THE INVENTION

The technical approaches of certain embodiments of the present invention are described in detail and in completeness with the figures in the following description.

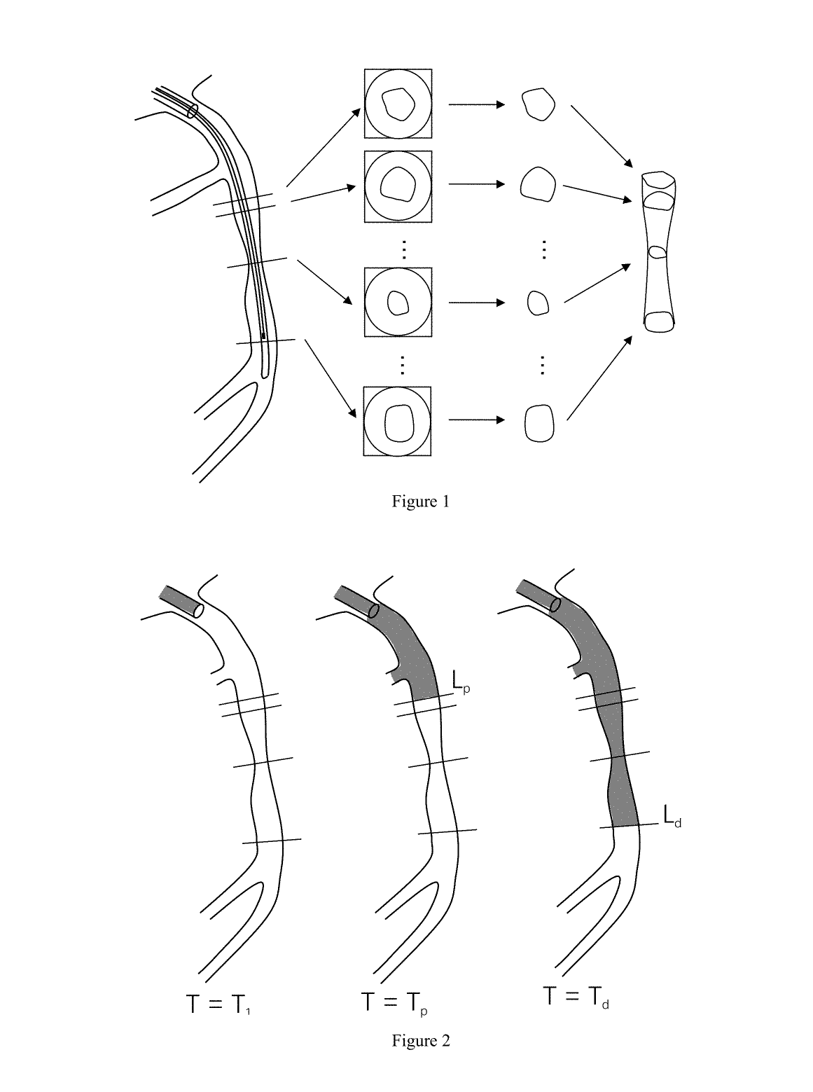

As illustrated in FIG. 1, a method to compute coronary indexes based on a high precision model, comprises acquisition of coronary angiography of coronary vessels, and intravascular images of vessels inside, and registration between coronary angiography and intravascular images into a high precision registration model, and calculation of coronary blood flow, fractional flow reserve and index of microcirculation resistance based on the high precision registration model.

Coronary angiography and intravascular imaging are two different approaches to estimate the disease severity of coronary arteries. Coronary angiography uses X-ray to generate projections of human body along a certain direction by injecting contrast through vessels, and the output is a projected two dimensional image with the maximum vessel diameter along this direction. Intravascular imaging uses an optical or ultrasound catheter to generate pipe-like circular images over all axial directions inside the vessel.

Coronary angiography and intravascular imaging complement each other for making a diagnosis of the stenosis of a diseased vessel. Coronary angiography has relatively low resolution, and has limited precision for quantifying the vessel diameter, area and stenosis, and is unable to differentiate between different atherosclerotic plaque types, but can provide the overall morphological information of coronary vascular trees. Intravascular imaging has higher resolution, and is able to compute the vessel area and stenosis precisely, and can effectively differentiate and make a diagnosis of atherosclerotic plaques inside the artery, but is unable to see the overall coronary vascular structures.

In summary, both coronary angiography and intravascular imaging have certain limitations, and each of them alone cannot perform real precise measurement. Hence, the present invention proposes to use both coronary angiography and intravascular imaging, and methods for achieving a high precision registration between the two images.

The method to register the coronary angiography and intravascular images into a high precision registration model is described in detail as follows:

First, a radio-opaque marker is placed on the intravascular imaging catheter, and in the initial stage, by locating the positions of the radio-opaque marker and guide wire in the coronary angiography images, and the insert directions of the guide wire, the possible range of the pullback trajectory of the radio-opaque marker or the guide wire during the subsequent intravascular imaging procedure can be roughly estimated.

In the second step, the coronary angiography console is turned on, and contrast is injected through the vessels via a catheter, and after the contrast is released, the time-stamped videos of coronary angiography is acquired. By detecting the vessel locations in the coronary angiography images, precise reference of the location information of the radio-opaque marker in the coronary angiography can be obtained. In one embodiment, the vessel location detection can be performed using the eigenvalues of Hessian matrices or other filtering methods.

In the third step, the locations of the vessels and the guide wire determined from the previous steps provide a rough range of the possible radio-opaque marker positions. The next step is to precisely detect the radio-opaque marker pullback trajectory. One embodiment is to use a matched filter to detect the radio-opaque position in every frame of the coronary angiography. The matched filter can be designed based on the unique features of the radio-opaque marker from pre-acquired coronary angiography images. In another embodiment, an objective function is used to locate the radio-opaque marker, and the optimal trajectory in the time-stamped coronary angiography images is determined using graph-cuts or Markov chain or Bayesian methods by globally optimizing the accumulated objective function. Another embodiment is to select one or multiple frames of coronary angiography images after contrast injection and manually mark the radio-opaque marker positions, and determine the optimal pullback trajectory using livewire or intelligent scissor algorithms.

In the fourth step, using the optimal pullback trajectory determined from the previous step, registration between the intravascular images and coronary angiography is completed, and every frame of the intravascular images is matched to a location in the corresponding coronary angiography frame.

One embodiment of the method to compute the coronary blood flow based on the high precision registration model is to select a vessel segment from the high precision registration model, and measure the transit time of the contrast traveling through the vessel segment, and obtain the lumen volume of the vessel segment in the high precision registration model, and compute coronary blood flow using equation (1):

$$Q = \frac{V}{Δ T} \qquad ( 1 )$$

As illustrated in FIG. 1, the lumen volume V is calculated from the morphological parameters measured using intravascular imaging based on the high precision registration model.

As illustrated in FIG. 2, one embodiment of the method to measure the contrast transit time ΔT inside the vessel segment is to inject contrast at the proximal end of the vessel, and record the time of the first frame of the coronary angiography T₁, the time of the second frame T₂, and so forth. Then the contrast transit time is obtained as ΔT=T_{d}−Tₚ, where Tₚ is the contrast arriving time at the proximal end of the vessel segment, and T_{d} is the contrast arriving time at the distal end of the vessel segment. Based on the registration methods between coronary angiography and intravascular imaging described previously, the three dimensional locations of the vessel Lₚ and L_{d} in the intravascular images corresponding to the contrast leading edge at Tₚ and T_{d}, respectively, can be obtained. The lumen volume between Lₚ and L_{d} can be determined based on the three dimensional models of the vessel. The coronary blood flow Q determined from equation (1) can be used in subsequent calculations of pressure drop and FFR.

Fractional flow reserve is calculated using equation FFR=P_{d}/Pₐ, where the distal end pressure P_{d} of the target vessel is determined by subtracting the pressure drop ΔP from the proximal end pressure Pₐ. The pressure drop of a fluid after passing through a pipe consists of the pressure drop from friction alone the path, gravity, acceleration and local resistance. In normal vessels, the friction pressure drop is the dominant factor for laminar flow. Assume the vessel length is L, vessel dimeter is d, and blood viscosity is μ, blood flow is Q (obtained previously), according to the Poiseuille's law, the pressure drop along the path takes the following form:

$$Δ P = \frac{64 µ QL}{π {d}^{4}}$$

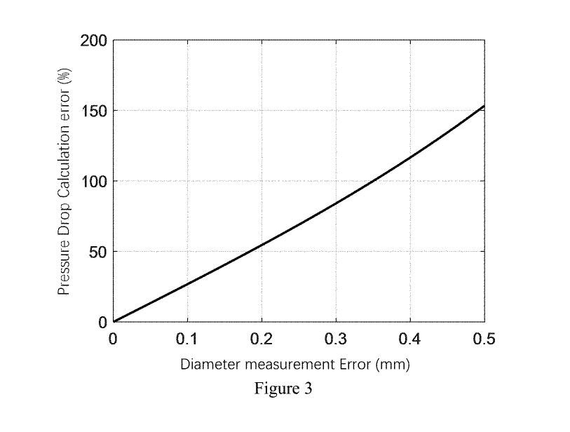

Therefore, in order to accurately calculate the pressure drop, it is necessary to precisely determine the blood flow Q, the vessel length L, and the vessel diameter d. In particular, the precision of vessel diameter d is of paramount importance. FIG. 3 illustrates the relationship between the computation errors of pressure drop and the measurement errors of diameter. The resolution of coronary angiography is around 0.5 mm, and the resulting computation errors of the pressure drop are significant, indicating that the result based on coronary angiography alone is unreliable. Intravascular imaging methods such as OCT with a resolution around 0.02 mm is able to control the computation errors of the pressure drop well. But because the penetration depth of OCT is limited, and blood clearance is required for imaging, it is sometimes challenging to acquire high quality images at all locations. On the other hand, IVUS does not require blood clearance during imaging, and the combination of OCT and IVUS can provide better intravascular imaging results.

Specifically, there are usually two ways to compute pressure drop. The first method is analytical, which divides the target vessel into small segments according to certain standard, and determine the overall pressure drop by summing over all the pressure drop from individual segments. The other method is numerical, based on computational fluid dynamic analysis, the pressure drop of the vessel segment is determined from calculating the pressure and flow of every unit volume inside the vessel using standard finite element analysis methods.

As illustrated in FIG. 2, the lumen volume between Lₚ and L_{d} and the pressure drop calculation method require intravascular imaging to accurately determine vessel area at each cross-section. One embodiment is to locate the frame locations of intravascular images between Lₚ and L_{d}, and perform segmentation of intravascular images and determine the lumen borders of the vessel in each frame, based on which reconstruction of the blood vessel model can be conducted, and pressure drop and FFR can be computed from the vessel lumen model utilizing the blood flow Q.

In one embodiment, the said index of microcirculation resistance is determined from fractional flow reserve.

In one embodiment, the said index of microcirculation resistance is computed from equation (2):

$$IMR = FFR \times \frac{{P}_{a}}{Q} \qquad ( 2 )$$

Where FFR is the fractional flow reserve, Pₐ is the mean arterial pressure, Q is the blood flow.

When there is coronary collateral flow that can not be neglected, calculation of IMR should be corrected using equation (3):

$${IMR}^{′} = \frac{{P}_{a} ( {P}_{d} - {P}_{w} )}{{Q}_{d} ( {P}_{a} - {P}_{w} )} = {FFR}_{cor} \times \frac{{P}_{a}}{{Q}_{d}} \qquad ( 3 )$$

Where P_{w} is the coronary wedge pressure, and is typically determined during coronary balloon angioplasty, or measured using a pressure wire at the distal end of the coronary artery after it is totally occluded. FFR_{cor} is the radio between the distal end pressure by considering only the stenosis of the coronary artery and the mean arterial pressure Pₐ. P_{d} is mean venous pressure.

The embodiments described above are only part, but not all, of the possible embodiments of the present invention. The embodiments based on the present invention, and all other embodiments generated by regular technical people in the relevant field without creative work, are within the scope of this invention.

1. A method to compute coronary physiology indexes, characterized in that comprising the acquisition of the coronary angiography images and intravascular images of coronary vessels, the registration of the coronary angiography images and the intravascular images into a high precision registration model, and the calculation of the coronary flow, the fractional flow reserve (FFR) and the index of microcirculation resistance (IMR) based on the high precision registration model.

2. The method of claim 1 for computing coronary physiology indexes based on the high precision registration model, wherein the method of performing co-registration of coronary angiography and intravascular images is realized by placing a radio opaque marker in the imaging catheter that moves together with the transducer, tracking the marker's position and pullback trajectory, locating the coronary vessel positions of the intravascular images in the corresponding coronary angiography, and generating a high precision registration model through signal synchronization and processing.

3. The method of claim 1 for computing coronary physiology indexes based on the high precision registration model, wherein the method for the computation of coronary blood flow based on the high precision registration model comprise selecting a segment of vessel from the high precision registration model, measuring the transit time of the contrast traveling through the vessel segment, calculating the vessel segment volume, and calculating the coronary flow using equation (1):
$$Q = \frac{V}{Δ T} \qquad ( 1 )$$
where Q is the coronary blood flow, ΔT is the contrast transit time, and V is the lumen volume.

4. The method of claim 3 for computing coronary physiology indexes based on the high precision registration model, wherein the vessel segment volume V is calculated from the vascular shape obtained by the intravascular images based on the high precision registration model.

5. The method of claim 3 for computing coronary physiology indexes based on the high precision registration model, wherein the method of measuring the contrast transit time ΔT for the vessel segment comprises injecting contrast from the proximal end of the vessel, recording the time of the first frame of the coronary angiography T₁, the time of the second frame T₂, and so forth, calculating the contrast transit time as ΔT=T_{d}−Tₚ, where Tₚ is the contrast arriving time at the proximal end of the vessel segment, and T_{d} is the contrast arriving time at the distal end of the vessel segment.

6. The method of claim 1 for computing coronary physiology indexes based on the high precision registration model, wherein the said fractional flow reserve is computed from the coronary blood flow and vessel morphological parameters measured based on the high precision registration model.

7. The method of claim 1 for computing coronary physiology indexes based on the high precision registration model, wherein the said index of microcirculation resistance is derived from the fractional flow reserve.

8. The method of claim 7 for computing coronary physiology index, where the index of microcirculation resistance is calcualted from equation (2):
$$IMR = FFR \times \frac{{P}_{a}}{Q} \qquad ( 2 )$$
where FFR is fractional flow reserve, Pₐ is mean arterial pressure, Q is blood flow.

9. The method of claim 1 for computing coronary physiology indexes based on the high precision registration model, wherein the intravascular images comprise intravascular ultrasound images, intravascular optical coherence tomography images, and combined use of intravascular ultrasound images and intravascular optical coherence tomography images.
